(19) United States
(12) Patent Application Publication
(10) Pub. No.: US 2025/0135127 A1
(43) Pub. Date: May 1, 2025

(54) MEDICAL INSTRUMENT FOR INTRODUCING A FLUID THERAPEUTIC SUBSTANCE INTO A CAVITY OF A BODY

(71) Applicant: Capnopharm GmbH, Tübingen (DE)

(72) Inventor: Wiebke SOLASS, Tübingen (DE)

(21) Appl. No.: 18/837,685

(22) PCT Filed: Feb. 24, 2023
(86) PCT No.: PCT/EP2023/054687

§ 371 Date: Aug. 12, 2024

(30) Foreign Application Priority Data
Feb. 25, 2022 (DE) 10 2022 104 560.9

(51) Int. Cl. A61M 11/00

(52) U.S. Cl. CPC A61M 11/001

(57) ABSTRACT

The invention relates to a medical instrument (100) for introducing a fluid therapeutic substance into a cavity of a body, the medical instrument (100) comprising the following: a nozzle (110) having a nozzle head (111) for nebulising the fluid therapeutic substance to be introduced; an at least substantially elongate and at least substantially rigid shaft (120), at one end (121) of which the nozzle head (111) is disposed; and a flexible hose (140) disposed within the elongate shaft (120) and fluidically connected to the nozzle head (111), the nozzle (110) having an impact device (130) comprising an impact body (131) disposed in front of a nozzle opening (112) of the nozzle head (111) at least sub-stantially coaxially with the nozzle opening (112).

### TECHNICAL FIELD

The invention relates to a medical instrument for introducing a fluid therapeutic substance into a cavity of a body. The medical instrument comprises a nozzle having a nozzle head for nebulising the fluid therapeutic substance to be introduced. Furthermore, the medical instrument comprises an at least substantially elongate and at least substantially rigid shaft, at one end of which the nozzle head is disposed. Furthermore, the medical instrument has a flexible hose disposed within the elongated shaft and fluidically connected to the nozzle head.

### PRIOR ART

Medical instruments for introducing a fluid therapeutic substance into a body cavity are known in the prior art. For example, DE 10 2012 104 629 A1 or WO 2020/048627 A1 disclose such instruments having a shaft, at one end of which a nozzle is disposed, by means of which the fluidtherapeutic substance can be sprayed into a cavity of a body. However, the structure of the known instruments is relatively complex. In addition, the fluid-like therapeutic substance cannot be optimally distributed in the body cavity by means of the known instruments. In addition, these technologies usually require a gas flow, which complicates the application in a closed body cavity: in order to avoid a pressure increase in the body cavity, an inlet and an outlet must be created, which makes it difficult to determine the proportion of a therapeutic solution that remains in the body cavity or only passes through this body cavity. In addition, it is not possible to check that the nebulisation system is closed. This means that the escape of a toxic substance (e.g. cytotoxic drugs) cannot be ruled out if the gas flow is continuous. Another problem in the state of the art is the occasionally high dead volumes in the delivery systems, which can result in 5% to 10% of the therapeutic substance not reaching the patient. There is also a need for improvement here.

### SUMMARY OF THE INVENTION

The object of the invention is to provide a medical instrument for introducing a fluid therapeutic substance into a cavity of a body, which eliminates the above-mentioned problems and disadvantages of the prior art. In particular, it is the object of the present invention to provide a medical instrument having a particularly simple structure and providing a particularly good distribution of the therapeutic substance in the body cavity.

The object is achieved by the subject-matter of independent claim 1. Further possible embodiments of the invention are disclosed in particular in the dependent claims.

In particular, the solution according to the invention consists in providing a medical instrument for introducing a fluid therapeutic substance into a cavity of a body, comprising the following: a nozzle having a nozzle head for nebulising the fluid therapeutic substance to be introduced; an at least substantially elongate and at least substantially rigid shaft, at one end of which the nozzle head is disposed; and a flexible hose disposed within the elongate shaft and fluidically connected to the nozzle head, wherein the nozzle comprises an impact device comprising an impact body disposed in front of a nozzle opening of the nozzle head at least substantially coaxially with the nozzle opening.

A body cavity is understood to be any cavity of the body itself, such as a natural cavity of a hollow organ or a cavity such as an abdominal cavity or pleural cavity, as well as any surgically created cavity, such as a pseudopneumoperitoneum. Hollow organs such as the oesophagus, stomach, urinary bladder or other organs that contain a virtual space and are lined with an epithelium, for example, can often serve as a cancer focus. The abdominal cavity and the pleural cavities are particularly important regions of the body with regard to cancer foci. The abdominal space extends from the thorax at the thoracic diaphragm to the pelvis at the edge of the pelvis. The abdominal space is known as the abdominal cavity and houses numerous organs such as the stomach, small and large intestine, liver, gallbladder and pancreas. The abdominal cavity and the pleural cavities are lined by an extensive membrane called the peritoneum or pleura, which covers the abdominal wall and the pelvic walls (parietal peritoneum) and the organs contained therein (visceral peritoneum) or the inner surface of the chest cavity (parietal pleura) and the organs contained therein (visceral pleura). For example, a cancer site can spread in these regions, i.e. the respective tissue or part of the tissue in which cancer cells are present can be found there.

The fluid therapeutic substance is a fluid, in particular a pharmaceutical, which is introduced into the cavity of the body of the patient to be treated as part of a therapeutic measure or treatment of a patient. The fluid, preferably a pharmaceutical, is particularly liquid and not gaseous. Preferably, only the fluid (liquid) therapeutic substance is nebulised, without the addition of gas. This means that only a vapourised liquid escapes from the nozzle and no gas. The fluid therapeutic substance is primarily used to treat the cancer site. Preferably, the fluid therapeutic substance is or comprises one or more chemotherapeutic agents. Preferably, the one, two, more or all chemotherapeutic agent(s) used in the present invention is/are selected from the group consisting of chemotherapeutic agents used against a type of cancer selected from the group comprising: Gastrointestinal cancer, in particular stomach cancer, colon cancer, liver or pancreatic cancer, appendix cancer, oesophageal cancer, hepatocellular carcinoma, gynaecological cancer; in particular primary peritoneal cancer, ovarian cancer, endometrial cancer; prostate cancer, leukaemia, lymphoma, soft tissue sarcoma, multiple myeloma, bladder cancer, lung cancer, thyroid cancer, Kaposi's sarcoma and tumours of embryonic origin.

The volume of the active substance or of each active substance, preferably chemotherapeutic agent, but also DNA, RNA, nanoparticles, immunotherapeutic agents and viruses, is preferably contained in a volume of 5 ml to 2500 ml, preferably 10 ml to 1000 ml, more preferably 20 ml to 500 ml, more preferably 30 to 300 ml of fluid therapeutic substance, this information preferably referring to the total amount administered during an application, preferably dissolved or suspended or redispersed in hydrophilic or lipophilic solutions and emulsions.

Preferably, a treatment is directed to a type of cancer selected from the group comprising: Gastrointestinal cancer, in particular stomach cancer, colorectal cancer, hepatobiliary or pancreatic cancer, appendix cancer, oesophageal cancer, hepatocellular carcinoma, or gynaecological cancer; in particular primary peritoneal cancer, ovarian cancer, endometrial cancer, and tumours of embryonic origin. The treatment described here may be aimed at reducing tumour size and the number of tumour foci. Tumour foci include the primary tumour as well as metastases and other tumour proliferations. Treatment may also be aimed at preventing or delaying the recurrence of cancer after tumour removal or partial removal of the tumour, preferably after surgical tumour removal.

It is preferred that the treatment is repeated, with two, three or more administrations of the therapeutic substance aerosolised by means of the delivery system.

In particular, for the medical instrument or administration system, the end of the hose is connected directly to the nozzle. The other end of the hose is fluidically connectable or connected to a fluid source for providing the fluid therapeutic substance. The hose extends through the shaft to the end of the shaft where the nozzle is located. In this way, the “dead volume” of the therapeutic substance in the delivery system can be reduced to zero. Preferably, the hose is connected directly to the fluid source. However, the hose may also be connected to the nozzle only indirectly, in particular by inserting a connecting element. For example, it is also conceivable that the shaft itself forms a cavity that is connected to an outlet of the hose on the one hand and to an inlet of the nozzle on the other, so that the shaft itself is part of the fluid connection. In this case, however, the “dead volume” of the therapeutic substance that does not reach the patient may be increased.

In the delivery system, an impact device may preferably be disposed on the nozzle head. Furthermore, at least one part of the impact device is preferably formed in one piece with the nozzle head. The fact that the impact body is disposed in front of the nozzle opening of the nozzle head means that the impact body is disposed in a direction away from the shaft. The impact body is therefore disposed in such a way that the substance flowing out of the nozzle opening hits the impact body and is better distributed by means of the impact body.

On the one hand, the impact body ensures a particularly wide nebulisation angle. For example, a nebulisation angle greater than 120°, in particular greater than 150°, can be achieved.

On the other hand, the impact body does not change the particularly small droplet size. For example, a median aerodynamic droplet size (MAD—median aerodynamic diameter) of less than 35 μm, in particular less than 30 μm, can be achieved. This droplet size hardly changes between water (aqua distillata), sugary solutions (e.g. Glc 5%) and oily solutions (e.g. silicone oil 5). The viscosity of the fluid therapeutic substance is preferably in a range of 1 to 100 mPas, more preferably 1 to 30 mPas.

Overall, the impact device results in a particularly good distribution of the therapeutic substance in the cavity, while at the same time simplifying the design of the medical instrument and without impairing the function of the cone nozzle.

According to an advantageous refinement of the invention, the impact body has a parabolic shape, a triangular shape, a trapezoidal shape or a stepped pyramid shape in longitudinal section. The longitudinal section is a section along a cutting plane in which lies an axis of rotation of the instrument, which extends through the nozzle opening. The parabolic shape and the stepped pyramid shape are particularly suitable for distributing the substance well. Advantageously, the impact body is rotationally symmetrical, preferably about an axis that is coaxial with the nozzle opening. A parabolic shape then results in an ellipsoidal body, a triangular shape results in a cone, a stepped pyramid shape results in a stepped cone, wherein the steps may be cylindrical or also conical or frustoconical.

An advantageous refinement of the invention provides that the impact device has a holding device on which the impact body is disposed. The impact body can therefore be easily positioned at the correct position on the nozzle by means of the holding device. The holding device is preferably at least essentially thin in the form of a rod and is also preferably bent by 90° or 180°. Due to the rod-shaped design, the holding device only slightly hinders the distribution of the substance. The holding device can also comprise more than one arm, so that the individual arms can be thinner. In a further advantageous refinement of the invention, the holding device is L-shaped or U-shaped, wherein the impact body is disposed at a first end of the holding device, and wherein the second end of the holding device is attached radially on the outside to the nozzle head and/or to the shaft.

In particular, the holding device is designed as an L-shaped bent rod, the diameter of which is preferably a maximum of ⅕ of the diameter of the nozzle head. This means that the holding device blocks the distribution of the substance particularly little. If a plurality of rods are provided, the sum of the diameters of the two or more rods is preferably a maximum of ⅕ of the diameter of the nozzle head, preferably a maximum of 1/7, more preferably a maximum of 1/10.

Irrespective of this, the holding device may be tapered towards the nozzle opening. In particular, the holding device may have tapered deflection surfaces, whereby the deflection surfaces of the holding device deflect the substance so that the substance adheres less to the holding device.

An advantageous refinement of the invention provides that a charging device for electrostatically charging the fluid therapeutic substance is disposed on the holding device.

The electrostatic charging of the fluid therapeutic substance can improve the adhesion and/or the penetration depth of particles of the substance to or into tissue in the cavity.

In an advantageous refinement of the invention, the charging device is disposed on an opposite side to the impact body on the holding device.

In particular, a charging region of the charging device designed for electrostatic charging is disposed on the opposite side to the impact body on the holding device. This means that the charging device or the charging region is not in the direct spray field of the nozzle. This prevents the charging device or the charging region from being covered by the substance or deposits thereof. The charging device or the charging region may generally be brush-like or made of solid material. In a brush-like design, the charging region is formed with several electrical wires. In the case of a solid material design, the charging region has a uniform shape. The charging region may particularly preferably be flush with the holding device, particularly in the case of a uniform shape design.

According to an advantageous refinement of the invention, the charging device has a conductive foil or is formed from a conductive foil. Such a conductive foil can be understood as the implementation of the charging device or the charging region made of solid material. The conductive foil preferably extends from one end of the shaft, where the nozzle is located, along the shaft to the other end of the shaft located away from the nozzle. At the other end, the conductive foil can be connected or is connected to an energy source, in particular a high-voltage source, by means of a connection. At one end of the shaft, the conductive foil extends from the shaft along the nozzle or along the nozzle head and the holding device of the impact device.

The conductive foil makes it easy and safe to charge the substance at the charging region of the charging device. In addition, the conductive foil is extremely flat, so that the maximum size of the instrument, for example for use in a trocar, is not exceeded.

An advantageous refinement of the invention provides that a region of the conductive foil is exposed to form a charging region for electrostatically charging the fluid therapeutic substance.

In other words, an electrical conductor of the conductive foil is exposed at the charging region in such a way that it is configured to electrostatically charge the fluid therapeutic substance. The exposed region is therefore configured to emit an electrostatic charge to the therapeutic substance or particles thereof, in particular during treatment. For example, an insulating film of the conductive foil can be removed from the charging region so that the electrical conductor is exposed for contacting the substance.

In an advantageous refinement of the invention, the nozzle head has a plug-in connection at an end opposite the nozzle opening, with the hose being plugged directly onto the plug-in connection. As the hose is connected directly to the nozzle head or its plug-in connection, a simple instrument is achieved in which only a single connection point is required to guide the therapeutic substance to the nozzle head. This minimises the number of connection points and thus the resulting weak points, such as leaks or pressure loss. Furthermore, the substance only comes into contact with the nozzle and the hose, so that contamination can be minimised. In addition, the plug-in connection can be sealed without the need for expensive sealants. Sealing is particularly easy if the hose is pushed onto the plug-in connection with a press fit. The hose is therefore preferably flexible in such a way that it expands radially when pushed onto the plug-in connection. To make it easier to push on, the plug-in connection may preferably have a bevelled edge on its outer circumference facing the hose. It is also easier to provide different variants of the medical instrument customised for specific substances.

The nozzle head may be formed in one piece with the plug-in connection. In this case, it also has a continuous, preferably straight bore for conducting the fluid therapeutic substance. Alternatively, however, it would be conceivable for the nozzle head to be made up of several pieces, for example two. Additional functional elements may then simply be integrated into the nozzle. In a two-piece variant, in which the plug-in connection and the rest of the nozzle head are in two individual parts, the plug-in connection may preferably be pressed into the nozzle head. It is particularly preferable for the plug-in connection and/or the nozzle head to have several radially circumferential sealing ribs. This means that no further sealing is required.

According to an advantageous refinement of the invention, the medical instrument has a clamping sleeve disposed on the hose in such a way that the clamping sleeve presses the inner circumferential surface of the end of the hose plugged onto the plug-in connection against the outer circumferential surface of the plug-in connection. This allows the hose to be held on the plug-in connection in a sealing manner. The clamping sleeve creates a counterpressure on the plugged-on end of the hose from the outside so that the end of the hose sits tightly on the plug-in connection.

An advantageous refinement of the invention provides for the shaft to have a preferably circumferential stop region against which the clamping sleeve abuts. The stop region allows the position, in particular the axial position—seen in the direction of the axis of rotation—of the clamping sleeve to be specified precisely. At the same time, the stop region ensures that the clamping sleeve is prevented from moving, particularly axially.

Preferably, the nozzle also has a corresponding additional stop region. In the assembled state of the instrument, the clamping sleeve is then disposed in particular axially between the stop region of the shaft and the further stop region of the nozzle.

In other embodiments, the hose is inserted into the nozzle head. For this purpose, the nozzle head may have a hose holder opening into which the hose can be inserted. For example, the hose may also be connected to the nozzle head using suitable welding processes. Screwing and bonding can be avoided, which improves safety and also simplifies production.

In a further advantageous refinement of the invention, the medical instrument has a filter or particle filter preferably disposed in the nozzle. The filter or particle filter may be a component designated in advance as a functional element. Accordingly, the nozzle may then have a two-piece design in particular. The particle filter may be a polypropylene or metal filter, for example. The filter is disposed in particular to clean the fluid therapeutic substance of impurities. Alternatively, it would also be conceivable for the filter to be disposed in the hose, preferably at the end of the hose facing the nozzle head. However, it may also be disposed on the inlet side of the hose or be an integral part of the hose.

According to an advantageous refinement of the invention, a bore in the shaft is designed as a clearance fit to an outer diameter of the hose in such a way that the hose is disposed movably, in particular rotatably, within the shaft. This improves the handling of the medical instrument, as the hose can rotate as well.

In a further advantageous refinement of the invention, the medical instrument further comprises an adjustable outer stop for positioning in a trocar and a handle disposed on the shaft. The outer stop makes it easy to ensure correct positioning within the trocar. The handle improves the handling of the medical instrument. A discharge region may preferably be integrated on the outer stop, which for example has a discharge connection for connecting to a pipe. The discharge region is used, for example, to safely discharge a toxic aerosol after the operation.

According to an advantageous refinement of the invention, the nozzle has a machinereadable identifier containing information on the type of nozzle. The information may, for example, be information on the substances that may be used with the nozzle. This means that different nozzles, which differ from other nozzles, for example in the size of the nozzle opening or the design of the impact device, may be suitable for use with different substances. The machine-readable identifier may be a chip or a barcode in particular.

For example, the nozzle head may be interchangeably disposed on the shaft and, in particular, detachably attached to the shaft by means of a thread and/or a latching device, preferably using a special tool. This means that several nozzles can be used with one instrument. Thus, this also results in a system for introducing a therapeutic substance into a cavity of a body, the system comprising: one of the medical instruments described above; and a further nozzle with a further nozzle head and a further impact device with a further impact body, wherein the further nozzle head has a further nozzle opening, wherein the further nozzle is attachable to the shaft instead of the nozzle, wherein the nozzle opening of the nozzle head is different from the further nozzle opening of the further nozzle head and/or the impact body of the nozzle head is different from the further impact body.

Even if the nozzle head has previously been described as replaceable and nondestructively detachable from the shaft for this purpose, in a further preferred embodiment the nozzle head may be firmly connected to the shaft and in particular not nondestructively connected. For example, the nozzle head may be pressed into the shaft. For this purpose, the shaft and/or the nozzle head may preferably have several radially circumferential sealing ribs, which are deformed accordingly during pressing.

The medical instruments according to the invention described above provide a simple yet safe tool for minimally invasive surgery and the administration of therapeutic aerosols into body cavities.

The instrument according to the invention is also provided in a system for introducing fluid therapeutic substances into cavities. The system preferably has a trocar, preferably a source for the therapeutic substance, preferably a fluid pump and one of the previously described instruments according to the invention, fluidically connected or connectable to the fluid source and the fluid pump, in particular by means of the hose, and can be introduced into the cavity by means of the trocar.

In a second consideration, the invention achieves the aforementioned object by a trocar and a medical instrument according to any one of the previously described preferred embodiments of a medical instrument according to the first consideration, wherein the medical instrument extends through the trocar.

In a third aspect, the invention solves the problem in a therapeutic method for treating tumour tissue in a hollow organ, by the steps of: Providing a trocar with a medical instrument accommodated therein according to one of the above described preferred embodiments of a medical instrument according to the first aspect of the invention; supplying a fluid therapeutic substance to the medical instrument; and nebulising the fluid therapeutic substance by means of the nozzle with a nebulisation angle of greater than 120°. The nebulisation angle is preferably greater than 150°, more preferably greater than 170°, 180°, 200°, 240°, 270°.

Preferably, only the fluid therapeutic substance is supplied to the medical instrument. This means, in particular, that no gas and/or no driving and/or carrier liquid is added to the medical instrument.

Preferably, the fluid therapeutic substance is supplied at a pressure in a range of 10 bar to 25 bar, preferably 11 bar to 20 bar (measured upstream of the nozzle). This can favourably influence the nebulisation of the fluid substance.

In a further aspect, the invention solves the above-mentioned problem by using a medical instrument according to one of the above-described preferred embodiments of a medical instrument according to the first aspect of the invention for treating tumour tissue in a hollow organ.

### BRIEF DESCRIPTION OF THE DRAWINGS

The different and exemplary features described above may be combined with one another in accordance with the invention, insofar as this is technically expedient and suitable. Further features, advantages and embodiments of the invention are shown in the following description of examples of embodiments and with reference to the figures. Shown are:

FIG. 1 is a schematic perspective view of an exemplary medical instrument according to the present invention;

FIG. 2 is a sectional view of the medical instrument shown in FIG. 1;

FIG. 3 is a schematic enlarged view of a front section of the medical instrument shown in FIG. 1 and FIG. 2;

FIG. 4 is a sectional view of the enlarged region of the medical instrument shown in FIG. 3; and

FIG. 5 is a schematic application of a medical instrument according to the present invention.

### WAYS OF IMPLEMENTING THE INVENTION

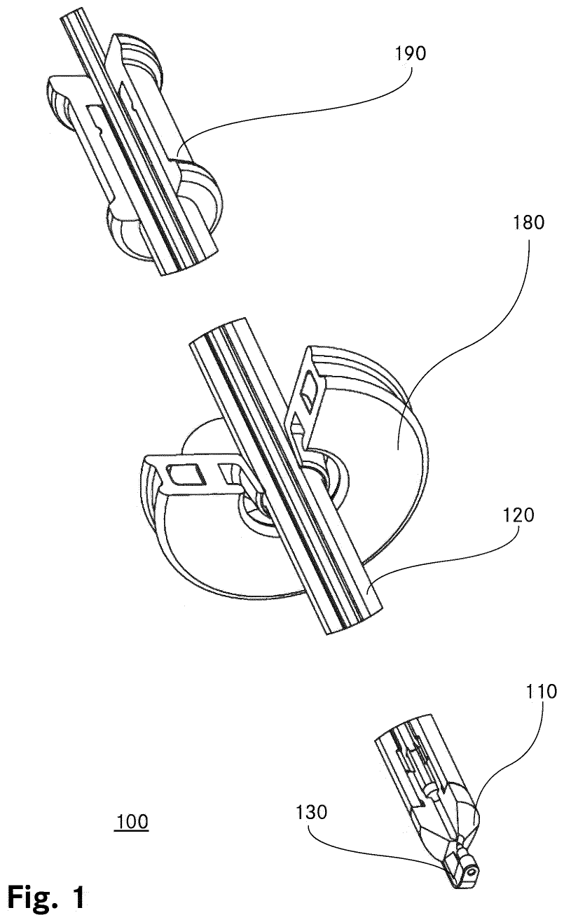

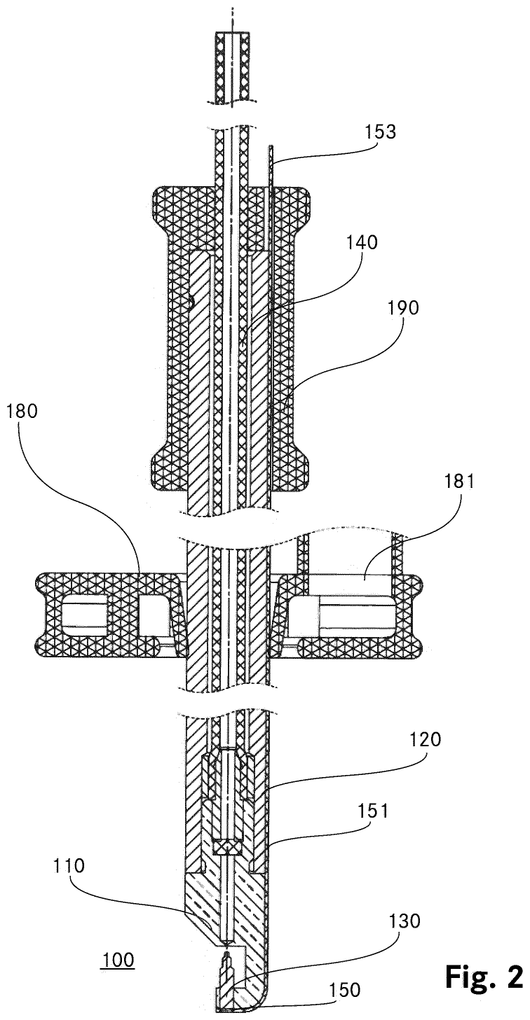

FIG. 1 shows a schematic perspective view of an exemplary medical instrument 100 according to the present invention. FIG. 2 shows a sectional view of the medical instrument 100 shown in FIG. 1.

As can be seen in FIG. 1 and FIG. 2, the medical instrument 100 has an elongated shaft 120. A nozzle 110 having an impact device 130 is formed at one end of the shaft 120. For a clearer presentation, the medical instrument 100 is shown using several breaks in order to have all aspects at a glance despite the length of the medical instrument 100. A handle 190 and an outer stop 180 are disposed on the shaft 120. The outer stop 180 serves as a stop for a trocar 200, not shown.

FIG. 2 shows that the outer stop 180 may perform a further function by means of a discharge region 181. For example, the outer stop 180 may also be used to discharge a fluid, such as insufflation gas, by means of the discharge region 181.

As can also be seen in FIG. 2, a hose 140 runs inside the shaft 120 as far as the nozzle 110. The hose 140 extends out of the shaft 120 at the end opposite to the nozzle 110 and may preferably be led to a source 400 of the therapeutic substance, not shown.

The charging device 150 comprises a conductive foil 151. The conductive foil 151 extends along the shaft 120 to the end opposite the nozzle 110. In particular, the conductive foil 151 extends along the outside of the shaft 120 to the opposite end. At this opposite end, for example, a connection 153 may be formed in order to connect the charging device 150 to an energy source 300, not shown, for providing the energy required for electrostatic charging.

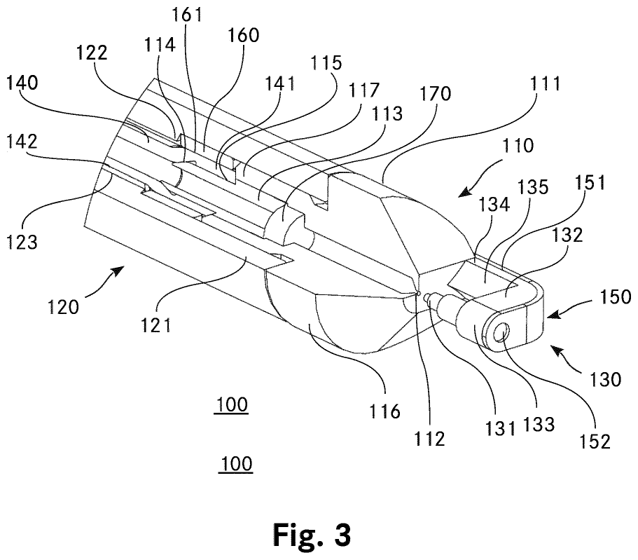

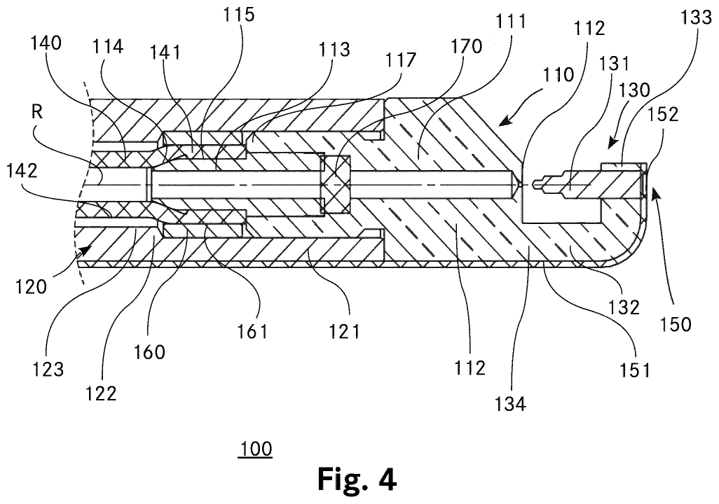

FIG. 3 shows a schematic, enlarged view of a front section of the medical instrument 100 shown in FIGS. 1 and 2. FIG. 4 shows a sectional view of the enlarged region of the medical instrument 100 shown in FIG. 3.

The nozzle 110 comprises a nozzle head 111. The nozzle head 111 is disposed at one end 121 of the shaft 120. Here, the nozzle head 111 is in two parts having a plug-in connection 113. Alternatively, a nozzle head 111 formed in one piece having a plug-in connection 113 would also be possible. The plug-in connection 113 is used to connect to the hose 140, in particular to one end 141 of the hose 140. For this purpose, the end 141 is pushed onto the plug-in connection 113. To make it easier to push on, the plug-in connection 113 has a bevelled edge 114. When the hose 140 or its end 141 is pushed on, it expands radially.

The nozzle head 111 further comprises a nozzle opening 112. The nozzle opening 112 is disposed at an end of the nozzle head 111 opposite the plug-in connection 113. The impact device 130 is also disposed at said opposite end. The impact device 130 has an impact body 131 serving to better distribute the substance. The impact body 131 is disposed coaxially to the nozzle opening 112, so that the fluid substance flowing out of the nozzle opening 112 strikes the impact body 131. Here, the impact body 131 has a stepped pyramid shape. Alternatively, this could also be parabolic, for example.

The nozzle opening preferably has a diameter in a range from 0.01 mm to 0.5 mm, preferably 0.02 mm to 0.4 mm, further preferably 0.05 mm to 0.3 mm.

The impact device 130 has a holding device 132 for arranging the impact body 131 in the correct position, i.e. coaxially to the nozzle opening 112. The holding device 132 has a first end 133, at which the impact body 131 is disposed, and a second end 134, at which the holding device 132 is connected to the nozzle head 111.

For example, as can be seen in FIG. 4, the holding device 132 may be formed integrally with the nozzle head 111. The holding device 132 extends generally from the nozzle head 111 first forwards in the axial direction (to the right in FIG. 4) and then inwards towards an axis of rotation R of the medical instrument 100, which also extends through the nozzle opening 112. As can be seen in FIG. 3, the holding device 132 may have one or more deflection surfaces 135 in order to deflect the substance and thus reduce deposition of the substance on the holding device 132.

Overall, the holding device 132 as shown in FIGS. 3 and 4, may be at least essentially L-shaped. Alternatively, the holding device 132 may also be U-shaped.

A part of the charging device 150 is also disposed on the nozzle 110 or on the nozzle head 111. In particular, a charging region 152 of the charging device 150 is disposed on the holding device 132. For example, as indicated in FIG. 3, the charging region 152 is an exposed region of the conductive foil 151 extending to the first end 133 of the holding device 132. The charging region 152 is disposed in particular on a side of the holding device 132 opposite the impact body 131. Thus, the charging region 152 is also disposed on a side of the holding device 132 facing away from the nozzle opening 112. This has the advantage that fewer particles of the fluid substance are deposited on the charging region 152, which could hinder the function of the charging device 150.

A particle filter 170 is also disposed inside the nozzle 110. In particular, the particle filter 170 is disposed between the plug-in connection 113 and the rest of the nozzle head 111.

Furthermore, the medical instrument 100 has a clamping sleeve 160 configured to press the hose 140 or its end 141 onto the plug-in connection 113. In particular, an inner circumferential surface 161 of the clamping sleeve 160 presses the hose 140 onto an outer lateral surface 115 of the plug-in connection 113. In the axial direction, the clamping sleeve 160 is disposed between a stop region 122 of the shaft 120 and a further stop region 117 of the nozzle 110. This can prevent the clamping sleeve 160 from slipping.

As can be clearly seen in FIG. 4, a clearance is formed between the hose 140 and the shaft 120 so that the hose 140 does not jam within the shaft 120. For this purpose, a bore 123 of the shaft 120 is larger than an outer diameter 142 of the hose 140.

The medical instrument 100 may have an identifier 116. In particular, the identifier 116 may be a machine-readable identifier containing information on the type of nozzle 110.

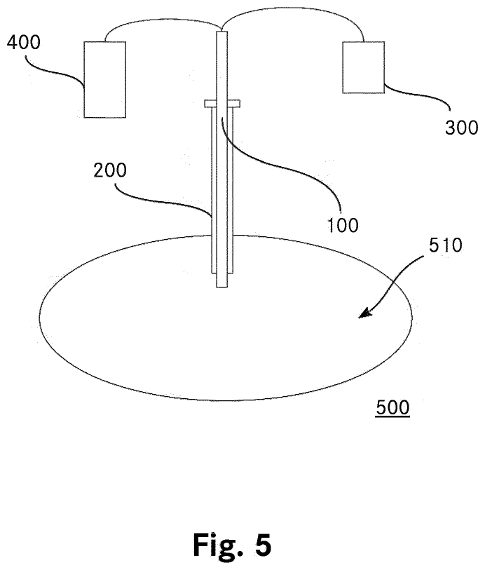

FIG. 5 shows the schematic application of the medical instrument 100 according to the present invention. The medical instrument 100 is configured to introduce a fluid therapeutic substance into a cavity 510 of a body 500. For this purpose, the medical instrument 100 may be disposed within the cavity 510 by means of a trocar 200. As indicated in FIG. 5, the medical instrument 100 is connected to a source 400 for providing the fluid therapeutic substance. Furthermore, the medical instrument 100 is connected to an energy source 300 for providing the energy for the charging device 150. The energy source is preferably configured to provide a voltage in a range from 4 kV to 9 kV, preferably 6 kV to 7 kV, and the charging device 150 is configured to receive such a voltage.

It should be noted that the features of the invention described with reference to individual embodiments or variants, such as the type and configuration of the individual components and their precise dimensions and spatial arrangement, may also be present in other embodiments. Furthermore, not all features of individual embodiments described in combination need necessarily be realised in a particular embodiment.

1. A medical instrument for introducing a therapeutic fluid into a cavity of a body, the medical instrument comprising:
a nozzle having a nozzle head configured to nebulize the therapeutic fluid to be introduced into the cavity;
a shaft, the shaft being at least substantially elongate and at least substantially rigid, the nozzle head being disposed on an end of the shaft; and
a flexible hose disposed within the shaft and fluidly connected to the nozzle head;
wherein the nozzle comprises an impact device having an impact body disposed in front of a nozzle opening of the nozzle head and substantially coaxial with the nozzle opening.

2. The medical instrument of claim 1, wherein the impact body has a parabolic, triangular, trapezoidal or pyramidal shape in a longitudinal section.

3. The medical instrument of claim 1, wherein the impact device comprises a holding device, the impact body being disposed on the holding device.

4. The medical instrument of claim 3, wherein the holding device is L-shaped or U-shaped;
wherein the impact body is disposed at a first end of the holding device; and
wherein a second end of the holding device is attached to at least one of the nozzle head or the shaft.

5. The medical instrument of claim 3, further comprising a charging device for electrostatically charging the therapeutic fluid disposed on the holding device.

6. The medical instrument of claim 5, wherein the charging device is disposed on a side of the holding device opposite the impact body.

7. The medical instrument of claim 5, wherein the charging device comprises a conductive foil.

8. The medical instrument of claim 7, wherein a region of the conductive foil is exposed to form a charging region for electrostatically charging the therapeutic fluid.

9. The medical instrument of claim 1, wherein the nozzle head has a plug-in connection at an end of the nozzle head opposite to the nozzle opening; and
wherein the hose is plugged directly onto the plug-in connection.

10. The medical instrument of claim 9, further comprising a clamping sleeve disposed on the hose, the clamping sleeve being configured to press an inner circumferential surface of an end of the hose against an outer circumferential surface of the plug-in connection.

11. The medical instrument of claim 10, wherein the shaft has a stop region abutting the clamping sleeve.

12. The medical instrument of claim 1, further comprising a particle filter disposed in the nozzle.

13. The medical instrument of claim 1, wherein the hose is movably disposed within a bore of the shaft.

14. The medical instrument of claim 1, further comprising an adjustable outer stop for positioning in a trocar and a handle disposed on the shaft.

15. The medical instrument of claim 1, wherein the nozzle comprises a machine-readable identifier containing information regarding a type of the nozzle.

16. A medical system comprising:
a trocar; and
a medical instrument comprising:
a nozzle having a nozzle head configured to nebulize a therapeutic fluid to be introduced into a cavity of a body;
a shaft, the shaft being at least substantially elongate and at least substantially rigid, the nozzle head being disposed on an end of the shaft;
a flexible hose disposed within the shaft and fluidly connected to the nozzle head;
an adjustable stop for positioning the trocar; and
a handle disposed on the shaft;
wherein the nozzle comprises an impact device having an impact body disposed in front of a nozzle opening of the nozzle head and substantially coaxial with the nozzle opening; and
wherein the medical instrument extends through the trocar.

17. A method for treating tumour tissue in a body cavity, the method comprising the steps of:
providing a trocar having a medical instrument comprising:
a nozzle having a nozzle head configured to nebulize a therapeutic fluid to be introduced into a cavity of a body;
a shaft, the shaft being at least substantially elongate and at least substantially rigid, the nozzle head being disposed on an end of the shaft; and
a flexible hose disposed within the shaft and fluidly connected to the nozzle head;
wherein the nozzle comprises an impact device having an impact body disposed in front of a nozzle opening of the nozzle head and substantially coaxial with the nozzle opening
delivering the therapeutic fluid to the medical instrument; and
nebulising the therapeutic fluid with the nozzle at a nebulisation angle of greater than 120°.

18. The therapeutic method of claim 17, wherein only the therapeutic fluid is supplied to the medical instrument.

19. The method of claim 17, wherein the therapeutic fluid is supplied at a pressure in a range from 10 bar to 21 bar.

20. (canceled)

21. The medical instrument of claim 1, wherein the impact body has a stepped pyramidal shape in a longitudinal section.
